(19) United States
(12) Patent Application Publication
(10) Pub. No.: US 2014/0309419 A1
(43) Pub. Date: Oct. 16, 2014

(54) METHOD FOR ENHANCING THE THERMAL STABILITY OF IONIC COMPOUNDS

(71) Applicants: Anders Riisager, Taastrup (DK); Rasmus Fermann, Copenhagen O (DK); Robin D. Rogers, Tuscaloosa, AL (US); Gabriela Gurau, Tuscaloosa, AL (US)

(72) Inventors: Anders Riisager, Taastrup (DK); Rasmus Fermann, Copenhagen O (DK); Robin D. Rogers, Tuscaloosa, AL (US); Gabriela Gurau, Tuscaloosa, AL (US)

(73) Assignees: Danmarks Tekniske Universite, Kgs. Lyngby (DK); The Board of Trustees of the University of Alabama for and on behalf of the University of Alabama, Tuscaloose, AL (US)

(21) Appl. No.: 14/241,685

(22) PCT Filed: Aug. 30, 2012
(86) PCT No.: PCT/EP2012/066898

§ 371 Date: Jun. 20, 2014

Related U.S. Application Data
(60) Provisional application No. 61/529,492, filed on Aug. 31, 2011.

(30) Foreign Application Priority Data
Aug. 31, 2011 (EP) 11179457.4

(51) Int. Cl. C07F 9/02; C07C 67/62; C07C 51/50; C07D 473/18

(52) U.S. Cl. CPC C07F 9/025; C07D 473/18; C07C 67/62; C07C 51/50
USPC 544/276; 560/151; 562/496; 562/17

(57) ABSTRACT

This invention relates to a method for enhancing the thermal stability of ionic compounds including ionic liquids, by immobilization on porous solid support materials having a pore diameter of between about 20-200 Å, wherein the solid support does not have a pore size of 90 Å.

### BACKGROUND

Ionic liquids are novel solvents of interest as greener alternatives to conventional organic solvents aimed at facilitating sustainable chemistry. As a consequence of their unusual physical properties, reusability, and eco-friendly nature, ionic liquids have attracted the attention of organic chemists. For example, the groups of Fehrmann and Wasserscheid introduced the concept of Supported Ionic Liquid Phase (SILP) catalysts for the immobilization of a transition metal catalyst dissolved in ionic liquids on solid carrier material (Riisager et al., Eur. J. Inorg. Chem. 2006, 695-706). In these SILP systems, a thin film of ionic liquid containing the homogeneous catalyst is immobilised on the surface of a high-area, porous carrier material. Consequently, SILP catalyst systems offer significant advantages compared to biphasic catalysis in organic liquid/ionic liquid mixtures. Examples of transition metal catalyzed reactions include hydroformylation, carbonylation, hydrogenation, Heck reactions, hydroaminations and epoxidation.

Numerous reports have revealed that many catalysts and reagents could be supported in the ionic liquid phase, resulting in enhanced reactivity and selectivity in various important reaction transformations. It has been generally assumed that ionic liquids are extremely stable compounds which due to their ionic nature have virtually no vapour pressure under temperatures deemed relevant for organic chemical reactions; it is however becoming evident from the increasing number of reports (see e.g., Chu et al. Molecules 2009, 14, 3780-3813) on the use of ionic liquids as solvents, catalysts, and reagents in organic synthesis that these are not totally inert under many reaction conditions. Methods for enhancing the stability of ionic liquids that would not interfere with the intended use of the liquid would therefore be highly interesting.

Another conceptually different interest in ionic liquids have arisen from the design of active pharmaceutical ingredients (APIs) in the form of ionic liquids, since liquid state properties can have profound impact on important properties for successful drug development (Hough et al., New J. Chem. 2007, 31, 1429-1436; Hough and Rogers, Bull. Chem. Soc. Jpn. 2007, 80, 2262-2269). Ionic liquid strategies can take advantage of the dual nature (discrete ions) of liquid salts to realize enhancements which may include controlled solubility (e.g., both hydrophilic and hydrophobic ionic liquids are possible), bioavailability or bioactivity, stability, elimination of polymorphism, new delivery options or even customized pharmaceutical cocktails (Rogers et al., WO 2007044693). However, liquid state properties also have significant negative impact on the ease of preparation and handling compared to solid drugs, and need special devices for dosing and administration.

The increased usage of ionic liquids for various purposes have thus demonstrated certain issues pertaining to their handling and stability which need to be addressed in order to further develop applications for these interesting compounds.

These issues have to some extent been addressed by the inventors of the present application in co-pending European Patent Application No. 10156242.9 filed in March 2010 which pertains to immobilizing pharmaceutically active liquids on solid support materials. The inventors reported herein that silica-supported ionic liquids can have improved thermal stability compared to the pure ionic liquid forms.

The inventors have now found that this phenomenon pertains in a broader sense to a range of ionic and ionizable compounds.

### SUMMARY OF THE INVENTION

The present inventors, who were also inventors of co-pending European Patent Application No. 10156242.9 filed in March 2010, reported therein that certain mesoporous silica-supported ionic liquids have improved thermal stability compared to the pure ionic liquid forms.

Porous materials are classified into several kinds by their size. According to the IUPAC notation (J. Rouquerol et al., “Recommendations for the characterization of porous solids (Technical Report)”. Pure & Appl. Chem. 1994, 66, 1739-1758), microporous materials have pore diameters of less than 20 Å (2 nm) and macroporous materials have pore diameters of greater than 500 Å (50 nm). A mesoporous material (as used in EP10156242.9) is a material containing pores with diameters between 20 Å and 500 Å (2 and 50 nm).

The inventors have since found that the thermal stability enhancement is not restricted to ionic liquids, but is applicable to other ionic compounds, and also to ionizable compounds such as free acids like carboxylic, sulphonic and phosphoric acids, which to a larger or smaller degree are dissociated into anions and cations when adsorbed on the porous support material. This enhancement is highly surprising since it has been reported that the presence of silica dramatically accelerates the thermal decomposition of ionic liquids ([Kosmulski et al., Thermochimica Acta 2004, 412, 47-53).

However, there are certain restrictions in applicability which were not known at the time of filing EP10156242.9.

First of all, the porous support material must have a pore diameter of less than about 200 Å (20 nm) to achieve the desired thermal stability enhancement. Porous materials having a pore diameter of about 400 Å or greater have no effect on the thermal stability of the adsorbed ionic compound, but otherwise work well for the purposes of EP10156242.9, which pertains to the simple immobilization of biologically active ionic liquids on mesoporous solid support materials, which have pore sizes up to 500 Å.

For the purpose of the present invention, however, porous materials having a pore diameter of about 200 Å or smaller, preferably 100 Å or smaller (i.e., on the borderline between micro- and mesoporous), seem to work best.

Secondly, as regards thermal stability enhancement, ionic compounds comprising anions which may act as hydrogen ion acceptors (e.g., oxyanions) seem to be affected most positively by adsorption on porous supports. Compounds comprising anions which cannot act as hydrogen ion acceptors, such as chloride, do not experience any significant enhanced thermal stability (as measured by TGA) when adsorbed on porous support, irrespective of the pore size.

In summary, ionic compounds which comprise anions which can partake in hydrogen bonding, including ionic liquids and ionisable compounds, when adsorbed on a porous solid carrier material such as silica and certain other inorganic, carbonaceous or polymeric carrier materials having a pore size of less than about 200 Å, are transformed into solid compounds with improved thermal stability of the ionic compound. A lower pore size limit of about 20 Å is recommended to ensure a sufficient diffusion rate in and out of the porous support.

Accordingly, the present invention in a first aspect provides the use of a porous solid support material having a pore size of between about 20-200 Å, for increasing the thermal stability of an ionic compound absorbed on said solid support.

In a second aspect, the present invention provides a method for enhancing the thermal stability of certain ionic compounds, which method comprises adsorption of said ionic compound on a porous solid support material having a pore size of between about 20-200 Å, such as silica and certain other inorganic, carbonaceous or polymeric carrier materials.

In a third aspect, the present invention as an alternative provides a method for raising the upper operating temperature of certain ionic compounds, which method comprises adsorption of said ionic compound on a porous solid support material having a pore size of between about 20-200 Å, such as silica and certain other inorganic, carbonaceous or polymeric carrier materials.

In a fourth aspect the present invention thus provides a method for enhancing the stability of certain ionic compounds towards oxidation, which method comprises adsorption of said ionic compound on a porous solid support material having a pore size of between about 20-200 Å, such as silica and certain other inorganic, carbonaceous or polymeric carrier materials, or combinations and mixtures thereof.

### BRIEF DESCRIPTION OF THE FIGURES

FIG. 1 depicts the thermal stability of supported vs. unsupported Choline Acyclovir with different loading of 10 or 20% (wt/wt) using thermogravimetrical analysis (TGA).

FIG. 2 depicts the thermal stability of supported vs. unsupported Trimethylhexadecylammonium acyclovir with different loading of 10, 20 or 50% (wt/wt) using thermogravimetrical analysis (TGA).

FIG. 3 depicts the thermal stability of supported vs. unsupported Dioctylsulfosuccinic Acid (Docusinic acid) with different loading of 10, 20 or 50% (wt/wt) using thermogravimetrical analysis (TGA).

FIG. 4 depicts the thermal stability of supported vs. unsupported Tetrabutylphosphonium Ibuprofenate with different loading of 10, 20 or 50% (wt/wt) using thermogravimetrical analysis (TGA).

FIG. 5 depicts the thermal stability of supported vs. unsupported Ibuprofen with a loading of 10% (wt/wt) using thermogravimetrical analysis (TGA).

FIG. 6 depicts the thermal stability of supported vs. unsupported Tetraethylammonium Glyphosate with different loading of 10, 20 or 30% (wt/wt) using thermogravimetrical analysis (TGA).

FIG. 7a depicts the thermal stability of supported vs. unsupported Butylmethylimidazolium acetate with a loading of 10% (wt/wt) on two types of porous silica: PG2000 (pore diameter 2000 Å) and PG500 (pore diameter 500 Å) using thermogravimetrical analysis (TGA).

FIG. 7b depicts the thermal stability of supported vs. unsupported Butylmethylimidazolium acetate with a loading of 10% (wt/wt) on two types of porous silica: PG75 (pore diameter 75 Å) and MCM-41 (pore diameter 50 Å) using thermogravimetrical analysis (TGA).

FIG. 8 depicts the thermal stability of supported vs. unsupported Butylmethylimidazolium chloride with a loading of 10% (wt/wt) on three types of porous silica: PG2000 (pore diameter 2000 Å), PG500 (pore diameter 500 Å) and PG75 (pore diameter 75 Å) using thermogravimetrical analysis (TGA).

FIG. 9 depicts the leaching studies of Tetrabutylphosphonium Ibuprofenate as a function of loading (10, 20, and 30% w/w), on one type of porous silica (pore diameter 90 Å)

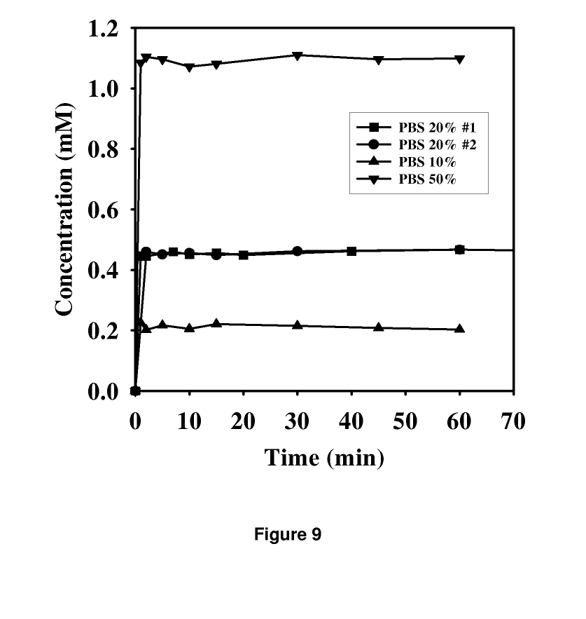

### DEFINITIONS

The term “ionic compound” as used herein comprises salts, ionic liquids and autoionizable chemical compounds such as acids which to a larger or smaller degree are dissociated into anions and cations when adsorbed on the porous support material.

The terms “autoionization” and “autoionizable” refer to the spontaneous separation of molecules into ions. Autoionization is also known as autodissociation.

The term “upper operating temperature” as used herein refers to the highest temperature to which the ionic compound can be heated without undergoing thermal degradation. This temperature is usually and routinely measured by Thermo Gravimetric Analysis (TGA) and/or Differential Scanning calorimetry (DSC). The decomposition temperatures were determined in isocratic TGA experiments either as the onset temperature for 5% decomposition T_{5% onset}, or the inflection point, i.e. the temperature at the inflection point of the TGA curve, which indicates the point where the degradation rate is maximum.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, hydroxyalkoxyalkyl, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OA¹ where A¹ is an alkyl group as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

### DETAILED DESCRIPTION OF THE INVENTION

The present invention is based upon the discovery that certain ionic compounds comprising anions which may act as hydrogen ion acceptors, for example oxyanion-based ionic liquids can be supported onto porous inorganic or carbon support materials such as silica which have a pore size of between 20 and 200 Å (2 and 20 nm), thereby being transformed into a solid compound with improved thermal stability of the absorbed ionic compound.

### Nature of the Supported Ionic Compound

Ionic compounds as referred to in the present invention include salts, ionic liquids and autoionizable chemical compounds such as acids, i.e., compounds which to a smaller or larger degree are dissociated into positively charged ions (cations) and negatively charged ions (anions).

Ionic liquids as referred to in the present invention is in liquid state at or below the human body temperature, preferably having a melting or glass transition point below 37 degree Celsius or even more preferably below 25 degree Celsius. In certain cases or for certain applications it may however be advantageous to employ ionic liquids having a melting or glass transition point above 37 degree Celsius.

The ionic compounds of the invention include a single compound or a mixture of two or more compounds, such as a eutectic mixture. Herein, the term “eutectic” means a mixture of two or more compounds which has a lower melting temperature than any of its individual compounds.

The ionic compound may contain one, two or more different components of which one or more may be ionic compounds such as salts. For example, in preferred embodiments of the invention, the cation is selected from differently substituted sulfonium, phosphonium or ammonium ions, or mixtures thereof, such as:

TABLE 1
Cations comprised by the invention

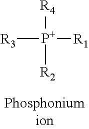

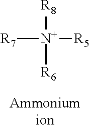

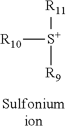

wherein R1, R2, R3, R4, R5, R6, R7, R8, R9, R10 and R11 can be, independently, hydrogen, alkyl, halogenated alkyl, aminoalkyl, alkenyl, alkynyl, aryl, heteroaryl, cycloalkyl, cycloalkenyl, heterocycloalkyl, or heterocycloalkenyl group as described above. The positively charged P, N and S atoms may also individually be part of heterocyclic or heteroaromatic structures by letting, e.g., R1 and R2 be fused such that a cyclic phosphonium ion is formed. Likewise, by letting eg. R5 and R6 be fused, a cyclic ammonium ion is formed, typical examples of which would be pyridinium and imidazolium. Finally, by letting eg. R9 and R10 be fused, a cyclic sulfonium ion is formed.

In other preferred embodiments the cation is heterocyclic or heteroaromatic containing 1-5 nitrogen atoms and 0-3 other heteroatoms selected from O or S, such as piperidinium, piperazinium, pyridinium, quinolinium, imidazolium, morpholinium.

Furthermore, in other preferred embodiments of the invention the anion is selected from substituted oxyanions such as carboxylates, phosphonates, phosphites, phosphonites, sulfonates, sulfinates and lactamates, including anions of certain nucleobases and their substituted analogues which may be formulated either with the negative charge placed on nitrogen or oxygen, such as the acyclovir anion:

TABLE 2
Anions comprised by the invention

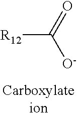

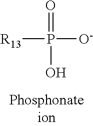

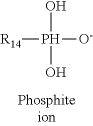

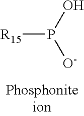

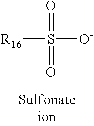

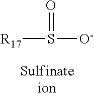

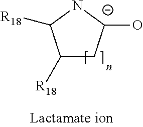

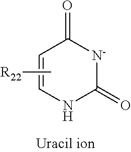

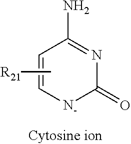

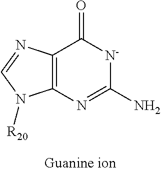

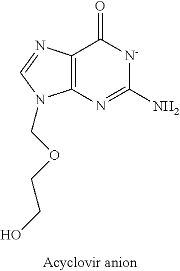

wherein n=1-3, R12, R13, R14, R15, R16, R17, R20 can be, independently, alkyl, halogenated alkyl, hydroxyalkoxyalkyl, alkenyl, alkynyl, aryl, heteroaryl, cycloalkyl, cycloalkenyl, heterocycloalkyl, or heterocycloalkenyl group as described above. R18 and R19 can be, independently, hydrogen, amino, alkyl, halogenated alkyl, alkenyl, alkynyl, aryl, heteroaryl, cycloalkyl, cycloalkenyl, heterocycloalkyl, or heterocycloalkenyl group as described above. R18 and R19 may also be fused such that a bicyclic, optionally heterocyclic ion is formed. R21 and R22 can be, independently, hydrogen, amino, alkyl, halogenated alkyl and halogen.

In preferred embodiments the ionic compound contains a cation as shown in Table 1 combined with one or more anions as shown in Table 2.

In other preferred embodiments the ionic compound contains one or more cations as shown in Table 1 combined with an anion as shown in Table 2. In yet other preferred embodiments the ionic compound contains one or more cations as shown in Table 1 combined with one or more anions as shown in Table 2.

In even more preferred embodiments, the ionic compound is selected from the compounds presented in Table 3.

### Adsorption on the Solid Carrier Material

The ionic compound is supported onto a solid carrier, or support material. The solid carrier material is substantially or completely insoluble in water, it is porous with a pore diameter of between about 20-200 Å, and it provides a medium to hold the ionic compound. The ionic compound is non-covalently adsorbed on its surface including the porous structure of the solid carrier material. The solid carrier material should preferably be a pharmaceutically acceptable and substantially non-toxic material, which can be any one of an inorganic, carbonaceous, and polymeric carrier material, having an acceptable porosity. In an embodiment of the invention, the porous solid support is selected from inorganic, carbonaceous or polymeric solid carrier materials. Preferably, the solid carrier material is mesoporous silica with a large surface area, a highly ordered pore structure and a pore size of about 20-200 Å. In alternate embodiments of the present invention, porous synthetic foam, porous ceramic, activated carbon, diatomaceous earth, zeolites, kieselguhr, charcoal, porous alumina, porous titania, porous zirconia or clay is employed. Mesoporous oxides of niobium, tantalum, cerium and tin may also be employed. Other carbon materials or layered double hydroxides can also be used as a solid carrier material for the ionic compound.

In a preferred embodiment of the invention, the solid carrier material is mesoporous silica with a pore diameter of between 20-200 Å.

The adsorption of an ionic compound on a particular solid carrier material is accomplished by dissolving the ionic compound in a suitable solvent and stirring the resulting solution with the solid carrier material for a sufficient period of time to allow equilibrium inside the pores to be established by pore diffusion (typically a couple of hours), evaporating the solvent slowly and removing the last traces of solvents in vacuo. In case the ionic compound is an ionic liquid, the resulting solid material is easier to handle than the ionic liquid itself, which can often be quite viscous, and can be prepared (“loaded”) with a high degree of precision.

The present invention thus provides a methodology to adsorb an ionic compound on a solid carrier material such as mesoporous silica having a pore diameter of about 20-200 Å to improve the thermal stability of the ionic compound.

The adsorption of ionic compounds on solid carrier materials according to the present invention in general takes place in a reversible or releasable manner, such that by placing the “loaded” carrier material in an aqueous environment such as, for example, simulated gastric fluid or simulated intestinal fluid, the supported ionic compound (including pharmaceutically active ionic compounds) are released rapidly and completely from the carrier material.

For liquid ionic compounds, one of the key benefits of supported ionic liquid phase (SILP) delivery systems is the ability to control and fine-tune the release of the adsorbed ionic liquid by adjusting the design of the ionic liquid form (i.e., the choice of anion and cation) of the active compound and/or by adjusting the solid carrier material. The flexibility of the supported ionic liquid phase (SILP) drug delivery technology thereby offers wide possibilities to design future tailor-made drug formulations. It has already been demonstrated in co-pending application EP10156242.9 that the rate of release of ionic liquids form the solid phase is very fast. The present invention now provides a protocol for the targeted development of novel SILP drug formulations having high stability towards thermal and oxidative degradation by prescribing both a range of pore diameters for the porous support and a suitable category of anions. Due to the porous structure of the support material, the adsorbed ionic compound can be obtained as a solid material even in high loading of 50% (wt/wt).

### Improved Stability of the Supported Ionic Compound

As discussed above, the adsorption of certain ionic compound on porous solid carrier materials with a pore diameter of about 20-200 Å has surprisingly been found to significantly enhance the thermal stability of the adsorbed compound compared to the pure (neat) compound.

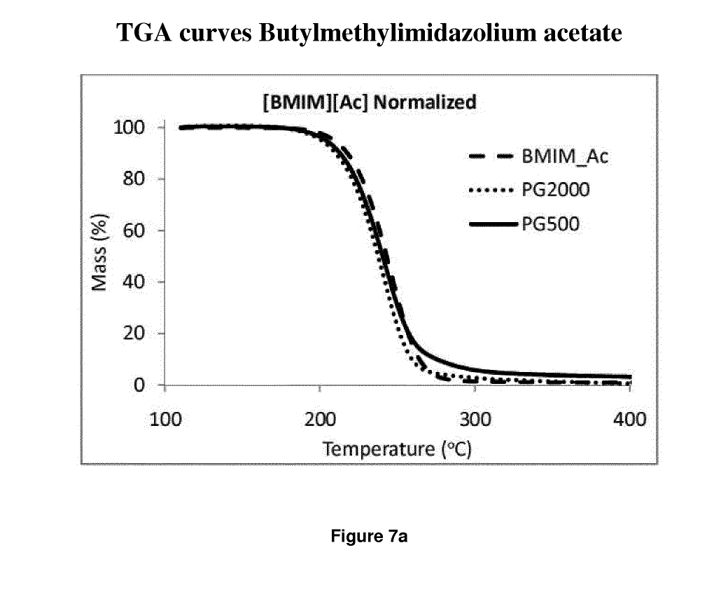

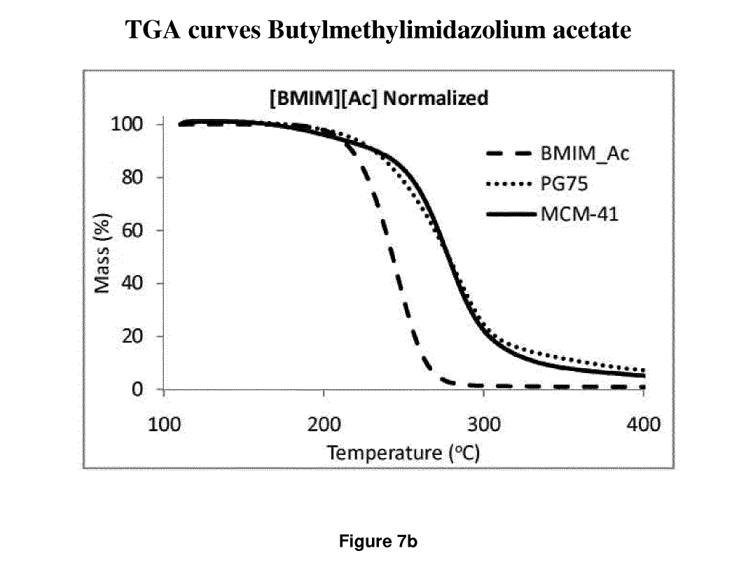

Most significantly, the pore diameter of the porous support has been found to have a great influence on the thermal stability enhancement. To test for such influence, adsorption experiments with butylmethylimidazolium acetate [BMIM][OAc] on four different porous silica types was carried out (FIG. 7a, 7b). The silicas had the following pore diameters: PG2000 (pore diameter 2000 Å), PG500 (pore diameter 500 Å), PG75 (pore diameter 75 Å) and MCM-41 (pore diameter 50 Å).

As can be seen from FIGS. 7a and 7b, no enhancement of thermal stability was observed on PG2000 (pore diameter 2000 Å), PG500 (pore diameter 500 Å), whereas adsorption on PG75 (pore diameter 75 Å) and MCM-41 (pore diameter 50 Å) led to significant enhancement of the thermal stability of the adsorbed [BMIM][OAc].

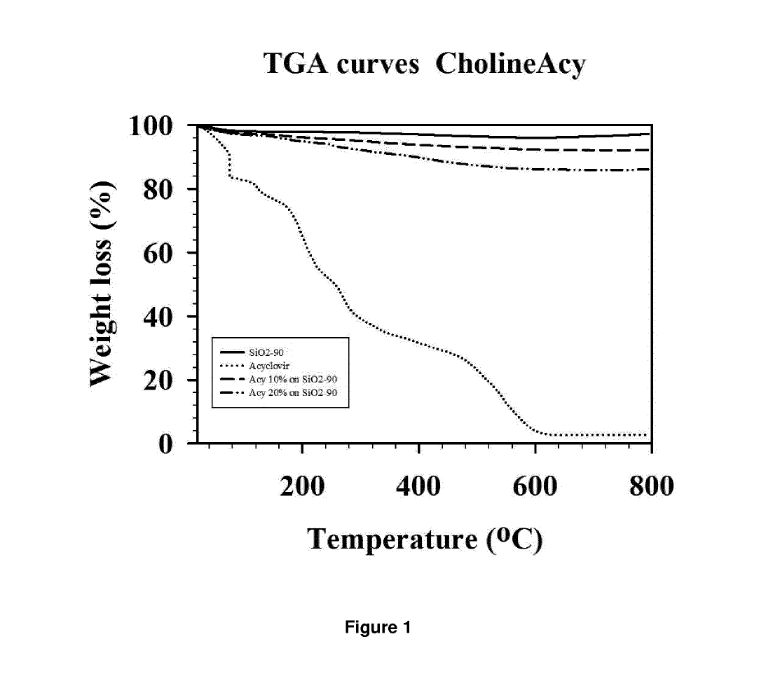

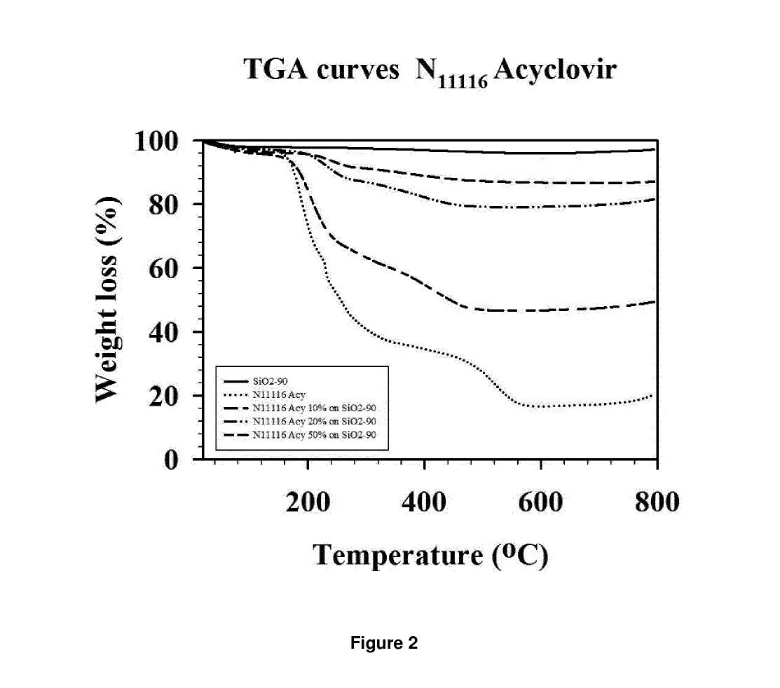

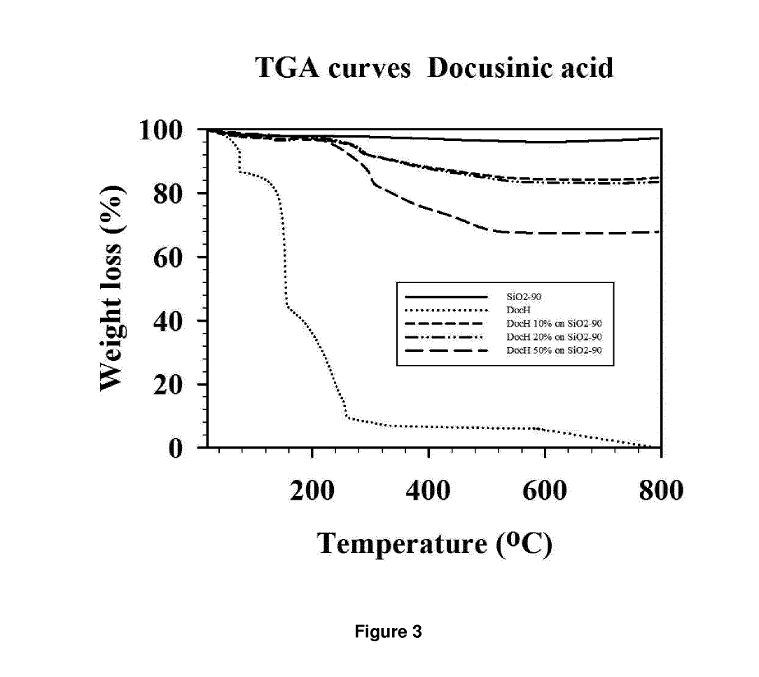

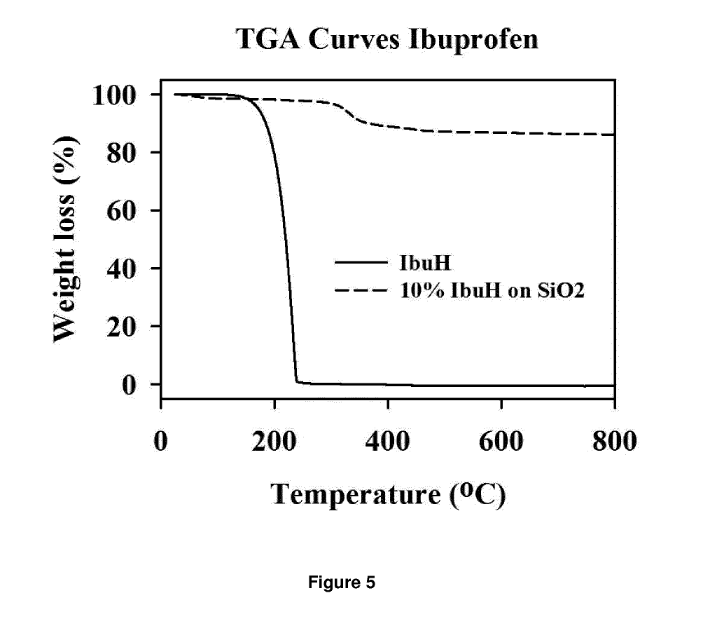

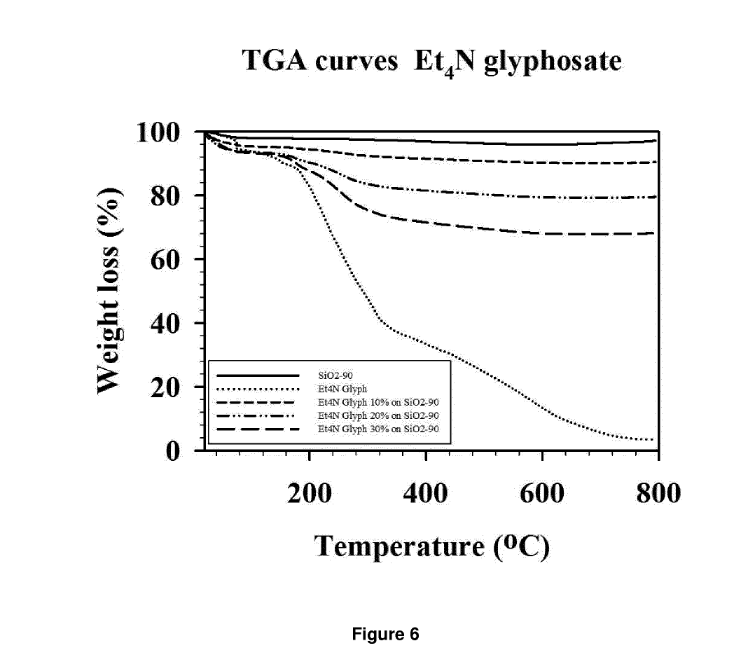

Adsorption of several other ionic compounds on SiO₂-90 (a 90 Å pore diameter porous silica) and subsequent TGA measurements also demonstrated significant enhancements of the thermal stability of the adsorbed ionic compounds (FIG. 1-6 and Table 3).

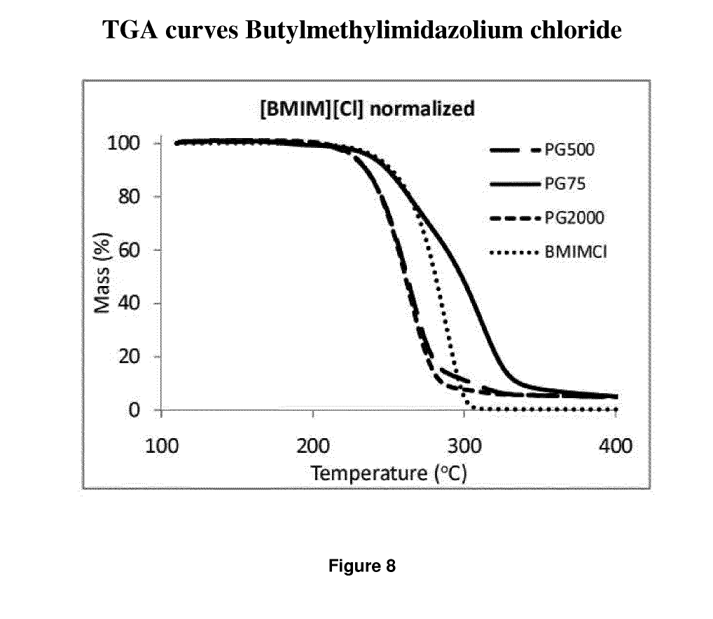

An influence of the anion could be observed when measuring the thermal stability of butylmethylimidazolium chloride [BMIM][Cl] adsorbed on three different porous silica types (FIG. 8). The silicas had the following pore diameters: PG2000 (pore diameter 2000 Å), PG500 (pore diameter 500 Å) and PG75 (pore diameter 75 Å). As can be seen from FIG. 8, no enhancement of thermal stability was observed on any of these support materials.

Without wishing to be bound by particular scientific theories, it is believed that the increased thermal stability of ionic compounds which includes anions capable of accepting hydrogen bonds such as oxyanions can be attributed to hydrogen bonding between the acidic sites on the inner surface of the porous structure and the adsorbed oxyanions. This type of bonding is not possible for e.g. chloride anions. The influence of the pore diameter, however, is very surprising and is presently being investigated.

The supported ionic compounds are also more resistant to oxidation than the unsupported compounds due to the relatively slow rate of oxygen diffusion through the porous structure of the support with pore diameters between 20-200 Å.

Accordingly, the present invention in a first aspect provides the use of a porous solid support material having a pore size of between about 20-200 Å, for increasing the thermal stability of an ionic compound absorbed on said solid support.

In a second aspect, the present invention provides a method for enhancing the thermal stability of certain ionic compounds, which method comprises adsorption of said ionic compound on a porous solid support material having a pore size of between about 20-200 Å, such as silica and certain other inorganic, carbonaceous or polymeric carrier materials.

In a third aspect, the present invention provides a method for raising the upper operating temperature of certain ionic compounds, which method comprises adsorption of said ionic compound on a porous solid support material having a pore size of between about 20-200 Å, such as silica and certain other inorganic, carbonaceous or polymeric carrier materials, or combinations and mixtures thereof.

The increased thermal stability is measured by Thermo Gravimetric Analysis under isocratic conditions and can be expressed conveniently by Δ_{T} which in the context of the present invention is the difference in thermal degradation temperature between the adsorbed ionic compound and the unsupported, neat ionic compound, and wherein the thermal degradation temperature in the context of the present invention is defined either as the onset temperature for 5% decomposition T_{5% onset}, or the inflection point, i.e., the temperature at the inflection point of the TGA curve, which indicates the point where the degradation rate is maximum.

An important effect of increasing the thermal stability of ionic compounds including ionic liquids is that the upper operating temperature of the ionic compound is raised thereby. It is known (see e.g., Chu et al. Molecules 2009, 14, 3780-3813) that many ionic liquids (Ls) contrary to common perception are not completely stable at higher temperatures. For certain applications, adsorption on a porous material having a pore size of between about 20-200 Å according to the present invention will raise the upper operating temperature of an ionic liquid sufficiently to carry out the desired operation involving the ionic liquid, or will protect the adsorbed ionic compound from long term effects of being stored at elevated temperatures.

In an embodiment of the invention, the thermal stability of the absorbed ionic compound is increased by Δ_{T}, wherein Δ_{T} is at least 5%, at least 10%, at least 15%, at least 20%, at least 25% or at least 30% of the thermal degradation temperature of the unsupported ionic compound.

In another embodiment of the invention the thermal degradation temperature of the absorbed ionic compound is increased by Δ_{T} relative to the unsupported, neat ionic compound, wherein Δ_{T} is at least 5%, at least 10%, at least 15%, at least 20%, at least 25% or at least 30% of the thermal degradation temperature of the unsupported ionic compound.

In yet another embodiment of the invention the thermal degradation temperature of the absorbed ionic compound is higher by Δ_{T} relative to the unsupported, neat ionic compound, wherein Δ_{T} is at least 5%, at least 10%, at least 15%, at least 20%, at least 25% or at least 30% of the thermal degradation temperature of the unsupported ionic compound.

In yet another embodiment of the invention the upper operating temperature of the absorbed ionic compound is higher by Δ_{T} relative to the unsupported, neat ionic compound, wherein Δ_{T} is at least 5%, at least 10%, at least 15%, at least 20%, at least 25% or at least 30% of the thermal degradation temperature of the unsupported ionic compound.

In preferred embodiments, Δ_{T} is at least 20%, at least 25% or at least 30% of the thermal degradation temperature of the unsupported ionic compound.

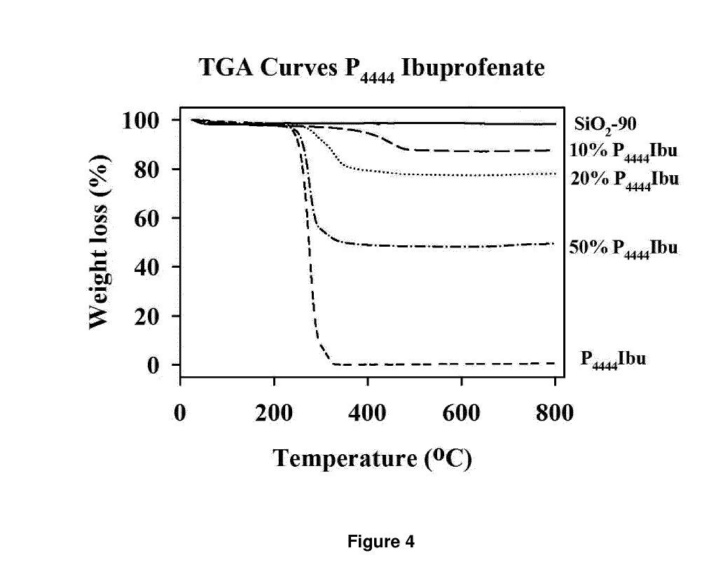

By way of example, the thermal degradation onset temperature of 10% (wt/wt) tetrabutylphosphonium ibuprofenate adsorbed on silica (T_{5% onset} 386.5° C.) is about 150° C. higher than the one of the pure ionic liquid tetrabutylphoshonium ibuprofenate (T_{5% onset} 236.6) (Table 3, FIG. 4). In this case ΔT=150° C. or an increase in thermal degradation temperature of over 60% of the thermal degradation temperature of the unsupported ionic compound.

In another preferred embodiment, the ionic compound comprises an oxyanion selected from carboxylates, phosphonates, phosphites, phosphonites, sulfonates, sulfinates and lactamates, including anions of certain nucleobases and their substituted analogues. In a specific embodiment, the ionic compound comprises the Acyclovir anion.

In a further preferred embodiment of the invention, the ionic compound is an ionic liquid. In an even more preferred embodiment, the ionic compound is an ionic liquid which comprises an oxyanion. In a further preferred embodiment, the ionic compound is an ionic liquid which comprises an oxyanion selected from carboxylates, phosphonates, phosphites, phosphonites, sulfonates, sulfinates and lactamates, including anions of certain nucleobases and their substituted analogues such as the Acyclovir anion.

In a further preferred embodiment of the invention, the ionic compound is selected from ammonium carboxylates, imidazolium carboxylates, pyridinium carboxylates, phosphonium carboxylates, ammonium phosphonates, imidazolium phosphonates, pyridinium phosphonates, phosphonium phosphonates, ammonium sulphonates, imidazolium sulphonates, pyridinium sulphonates, phosphonium sulphonates, ammonium acyclovirates, imidazolium acyclovirates and pyridinium acyclovirates.

In a particularly preferred embodiment of the invention, the ionic compound is selected from the following compounds: choline acyclovir and tetraalkylammonium acyclovir such as trimethylhexadecylammonium acyclovir.

The porous solid support material is preferably porous silica having a pore size of between about 20-200 Å. However, alumina and mesoporous oxides of niobium, tantalum, titanium, zirconium, cerium and tin having a similar pore size can also be utilized according to the present invention, as can certain other inorganic, carbonaceous or polymeric carrier materials, or combinations and mixtures thereof.

The transport/diffusion of gases into the porous structure of mesoporous support materials is to a large extent governed by the pore diameter of the porous structure [S. Satoh et al., Journal of Non-Crystalline Solids, 1995, 190, 206-211]. Oxygen gas diffusion in porous silica gel is thus limited by the average pore diameter of the gel. This means that an ionic compound adsorbed on a porous support is less likely to be attacked by oxygen on a support with relatively small pores than on a support with relatively large pores, and a lot less likely than the unsupported ionic compound.

In a fourth aspect the present invention thus provides a method for enhancing the stability of certain ionic compounds towards oxidation, which method comprises adsorption of said ionic compound on a porous solid support material having a pore size of between about 20-200 Å, such as silica and certain other inorganic, carbonaceous or polymeric carrier materials, or combinations and mixtures thereof.

Supported ionic liquids derivatives combine the advantages of ionic liquids with the advantages of a solid drug form. Specifically, the role of the ionic liquid is to eliminate polymorphism and to control and improve physical properties such as melting point, solubility and rate of dissolution of the solid active compound. Ionic compounds which are liquid at room temperature or slightly above have as a rule a higher solubility in aqueous media (including biological media) than crystalline ionic compounds due the lack of crystal lattice forces to be overcome. This is an advantage for e.g., drug molecules with limited solubility, which through conversion to an ionic liquid becomes more readily accessible. However, as the same crystal lattice forces also protect a crystalline compound from oxidative and/or thermal degradation, there are also drawbacks to converting crystalline compounds to ionic liquids.

The present invention presents a solution to this dilemma, as ionic liquids comprising oxyanions of the type discussed herein and adsorbed on porous support materials having a pore diameter of about 20-200 Å have significantly improved thermal stability over the unsupported ionic liquid. The high stability and easy handling normally associated with crystalline compounds can now according to the present invention be achieved simultaneously with the high solubility associated with amorphous or liquid forms of the compound.

In an embodiment of the invention the porous solid support material has a pore size of between 20-29 Å. In another embodiment of the invention the porous solid support material has a pore size of between 30-39 Å. In another embodiment of the invention the porous solid support material has a pore size of between 40-49 Å. In another embodiment of the invention the porous solid support material has a pore size of between 50-59 Å. In another embodiment of the invention the porous solid support material has a pore size of between 60-69 Å. In another embodiment of the invention the porous solid support material has a pore size of between 70-79 Å. In another embodiment of the invention the porous solid support material has a pore size of between 80-89 Å. In another embodiment of the invention the porous solid support material has a pore size of between 90-99 Å. In another embodiment of the invention the porous solid support material has a pore size of between 100-109 Å. In another embodiment of the invention the porous solid support material has a pore size of between 110-119 Å. In another embodiment of the invention the porous solid support material has a pore size of between 120-129 Å. In another embodiment of the invention the porous solid support material has a pore size of between 130-139 Å. In another embodiment of the invention the porous solid support material has a pore size of between 140-149 Å. In another embodiment of the invention the porous solid support material has a pore size of between 150-159 Å. In another embodiment of the invention the porous solid support material has a pore size of between 160-169 Å. In another embodiment of the invention the porous solid support material has a pore size of between 170-179 Å. In another embodiment of the invention the porous solid support material has a pore size of between 180-189 Å. In another embodiment the porous solid support material has a pore size of between 190-200 Å.

In a preferred embodiment of the invention the porous solid support material has a pore size of between 20 and 150 Å. In another preferred embodiment of the invention the porous solid support material has a pore size of between 40 and 100 Å.

In a particularly preferred embodiment of the invention the porous solid support material is mesoporous silica with a pore size of between 40 and 100 Å.

In another preferred embodiment the porous solid support material is mesoporous silica having a pore diameter selected from 50 Å, 75 Å and 90 Å.

The present invention will in the following section be exemplified by a number of non-limiting examples.

### EXAMPLES

### Example 1

### Synthesis of Choline Acyclovir [1]

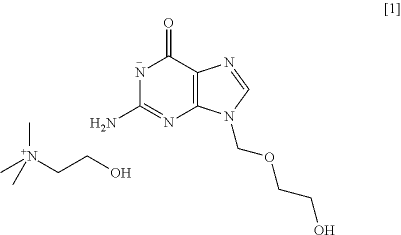

Acyclovir (0.693 mg, 3 mmol) was suspended in 20 mL of ethanol and choline hydroxide (3 mmol) 46% solution in water was added dropwise. The suspension was stirred for 15 min at room temperature until a clear solution was obtained and evaporated. Remaining volatile material was removed under reduced pressure (0.01 mbar, 50° C.) to yield choline acyclovir [1] as a colourless glass.

¹H-NMR (300 MHz, d₆-DMSO) δ (ppm)=7.4 (s, 1H), 5.2 (s, 2H), 4.9 (br s, 2H), 3.8 (s, 2H), 3.4 (m, 6H), 3.0 (s, 9H). ¹³C-NMR (75 MHz, d₆-DMSO) δ (ppm)=167.9, 161.8, 134.5, 118.9, 71.9, 70.4, 67.7, 60.3, 55.6, 53.5.

### Example 2

### Synthesis of Trimethylhexadecylammonium Acyclovir [2]

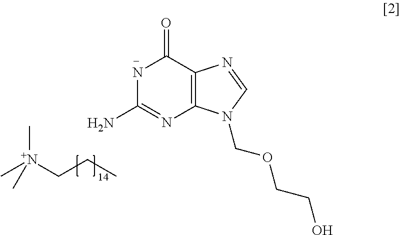

Prepared according to example 1 to give trimethylhexadecylammonium acyclovir [2] as a colourless solid.

¹H-NMR (300 MHz, d₆-DMSO) δ (ppm)=7.4 (s, 1H), 5.2 (s, 2H), 3.4 (s, 4H), 3.3 (m, 2H), 3.0 (s, 9H), 1.7 (m, 2H), 1.3 (s, 28H), 0.9 (t, J=7, 3H). ¹³C-NMR (75 MHz, d₆-DMSO) δ (ppm)=168.1, 161.9, 152.1, 134.2, 119.0, 71.9, 70.4, 65.6, 60.3, 52.5, 31.7, 29.4, 29.2, 29.1, 28.9, 26.1, 22.5, 14.3.

### Example 3

### Synthesis of Dioctylsulfosuccinic Acid [3]

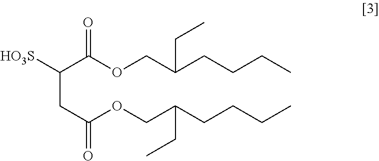 [SMILES: CCCCC(CC)COC(=O)CC(C(=O)OCC(CC)CCCC)S(=O)(=O)O]

Silver docusate [see Rogers et al., “Multi-functional ionic liquid compositions for overcoming polymorphism and imparting improved properties for active pharmaceutical, biological, nutritional, and energetic ingredients”, US 20070093462, Apr. 26, 2007] (10 g, 18.89 mmol) was suspended in 30 mL of methanol and HCl (37% solution in water; 1.56 mL, 18.89 mmol) was added dropwise. The suspension was stirred overnight at room temperature. The precipitate was filtered through Celite® and the filter cake was washed with additional 10 mL of cold methanol. The solvent was removed under reduced pressure (0.01 mbar, 50° C.) to yield dioctylsulfosuccinic acid [3] quantitatively as a light yellow viscous liquid.

¹H-NMR (300 MHz, d₆-DMSO) δ (ppm)=6.16 (br), 3.93-3.62 (m, 4H), 3.56 (s, 1H), 3.28 (d, 1H), 2.94-2.77 (m, 2H), 1.50 (br, 2H), 1.24 (br, 16H), 0.83-0.81 (m, 12H).

### Example 4

### Synthesis of Tetrabutylphosphonium Ibuprofenate [4]

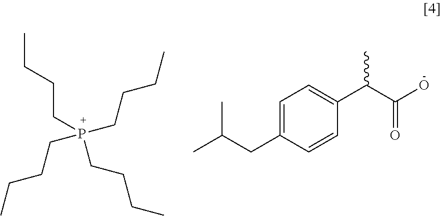

Ibuprofenic acid (1.032 g, 5 mmol) and tetrabutylphosphonium hydroxide (˜40% sol. in H₂O) (3.414 g, 5 mmol) were dissolved in 20 mL of acetone stirred for 15 min at room temperature. The solvent was evaporated and the remaining viscous liquid was dried at 0.1 mbar with stirring for 24 h to obtain tetrabutylphosphonium ibuprofenate [4] in quantitative yield as colourless viscous liquid. ¹H-NMR (300 MHz, d₆-DMSO) δ (ppm)=7.13 (d, J=8.08 Hz, 2H), 6.94 (d, J=8.08 Hz, 2H), 3.21 (q, J=7.74 Hz, 1H), 2.48 (m, 2H), 2.36 (d, J=7.28 Hz, 2H), 2.14 (m, 8H), 1.77 (sept, J=6.15 Hz, 1H), 1.40 (m, 16H), 1.18 (d, J=7.03 Hz, 3H), 0.91 (t, J=7.02 Hz, 12H), 0.84 (d, J=7.02 Hz, 6H). ¹³C-NMR (75 MHz, d₆-DMSO) δ (ppm)=174.8, 144.2, 136.9, 127.8, 127.2, 49.3, 44.4, 29.7, 23.4 (d, J=15.8 Hz), 22.7 (d, J=4.7 Hz), 22.2, 20.5, 17.3 (d, J=48.1 Hz), 13.3.

T_{g} −43° C., T_{5% onset} 236.6° C.

### Example 5

### Synthesis of Tetraethylammonium Glyphosate [5]

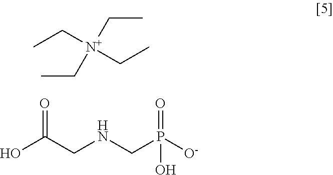

Tetraethylammonium chloride (1.66 g, 100 mmol) was suspended in 20 mL distilled water and NaOH (0.44 g, 110 mmol) dissolved in distilled water was added dropwise. AgNO₃ solution (1.7 g, 100 mmol dissolved in 20 mL distilled water) was added and the resulting mixture was stirred at 50° C. for 20 minutes. After cooling, the obtained solid was filtered and washed with distilled water. At this point, glyphosate (1.7 g, 100 mmol) was added and the reaction mixture was stirred at room temperature for 14 hours. Water was removed using a rotary evaporator and the obtained product [5] was dried under reduced pressure at 60° C. for 24 hours.

¹H-NMR (300 MHz, D₂O) δ (ppm)=4.9 (s, 3H), 3.73 (s, 2H), 3.28 (d, J=12.8 Hz, 2H), 3.22 (q, J=7 Hz, 8H), 1.25 (t, J=9.1 Hz, 12H). ¹³C-NMR (125 MHz, D₂O) δ (ppm)=173.6, 54.7, 47.8, 46.0, 9.4.

Butylmethylimidazolium acetate [BMIM][OAc] and Butylmethylimidazolium chloride [BMIM][Cl] were both commercially available.

### General Synthesis of Silica-Supported Compounds:

Silica was dried under heat (70° C.) and vacuum (0.01 mbar). API-IL (or starting API) was dried under vacuum and heat to remove volatiles or water and then weigh out ca. 0.01 g, and dissolved in suitable dry solvent (dry acetone or purchased anhydrous methanol or ethanol) to complete dissolution (˜20 mL of solvent). Silica (SiO₂) with a pore diameter between 20 Å and 2000 Å in an amount appropriate to target loading was suspended in solvent with dissolved API in it (20 mL) and stirred for 2 hours at room temperature. The solvent was evaporated (Rotovap) and sample kept under high vacuum (0.01 mbar) overnight.

### Example 6

### Thermal Stability of Silica-Supported Ionic Compounds

Thermal stability was measured by using thermogravimetric analysis (TGA), with isocratic heating at 5° C. min⁻¹ under an inert nitrogen and/or dried air atmosphere. The decomposition temperatures were determined from both the T_{5% onset} (onset temperature for 5% decomposition) in an isocratic TGA experiment, and from the inflection point (temperature at the inflection point of TGA curve, which indicates the point where the degradation rate is maximum). For T_{5% onset} measurements, thermal stability was determined in a Mettler-Toledo Star^{e} TGA/DSC unit by heating from 25° C. to 800° C. with a heating rate of 5° C./min under nitrogen. For inflection point measurements, thermal stability was determined using a TA2950 TGA unit by heating from 25° C. to 800° C. with a heating rate of 5° C./min under dried air.

TABLE 3
Improved thermal stability of supported ionic compounds

| | | Thermal |
|---|---|---|
| | | Stability |
| Compound | Loading | [° C.] |
| Choline Acyclovir [1] | not supported/neat | 122.51 |
| [1] on SiO₂ | 10% | 166.91 |
| [1] on SiO₂ | 20% | 165.40 |
| Trimethylhexadecylammonium | not supported/neat | 188.60^{b} |
| acyclovir [2] | | |
| [2] on 90 Å SiO₂ | 10% | 241.07^{b} |
| [2] on 90 Å SiO₂ | 20% | 233.51^{b} |
| Dioctylsulfosuccinic Acid [3] | not supported/neat | 161.86^{b} |
| [3] on 90 Å SiO₂ | 10% | 232.5^{b} |
| [3] on 90 Å SiO₂ | 20% | 217.87^{b} |
| Tetrabutylphosphonium | not supported/neat | 236.6^{a} |
| Ibuprofenate [4] | | |
| [4] on 90 Å SiO₂ | 10% | 386.5^{a} |
| [4] on 90 Å SiO₂ | 20% | 263.7^{a} |
| Ibuprofene (free acid) | not supported/neat | 154.7^{a} |
| Ibuprofene (free acid) on 90 Å SiO₂ | 10% | 300.0^{a} |
| Tetraethylammonium Glyphosate [5] | not supported/neat | 149.76^{b} |
| [5] on 90 Å SiO₂ | 10% | 178.51^{b} |
| [5] on 90 Å SiO₂ | 20% | 175.99^{b} |

^{a}measured using T_{5% onset} method;
^{b}measured using inflection point method

1. A method of enhancing the thermal stability of an ionic compound, the method comprising absorbing said ionic compound on a porous solid support material having a pore size of about 20-200 Å, provided the solid support does not have a pore size of 90 Å, said ionic compound comprising an anion that act as a hydrogen on acceptor.

2. The method according to claim 1, wherein the ionic compound comprises one or more anions selected from: 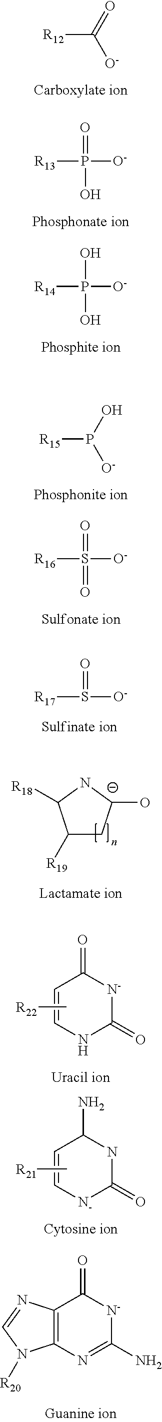 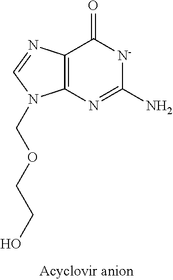
wherein
n=1-3,
R12, R13, R14, R15, R16, R17, R20 can be, independently, alkyl, halogenated alkyl, hydroxyalkoxyalkyl, alkenyl, alkynyl, aryl, heteroaryl, cycloalkyl, cycloalkenyl, heterocycloalkyl, or heterocycloalkenyl group,
R18 and R19 can be, independently, hydrogen, amino, alkyl, halogenated alkyl, alkenyl, alkynyl, aryl, heteroaryl, cycloalkyl, cycloalkenyl, heterocycloalkyl, or heterocycloalkenyl group,
R18 and R19 may be fused such that a bicyclic, optionally heterocyclic on is formed,
R21 and R22 can be, independently, hydrogen, amino, alkyl, halogenated alkyl and halogen.

3. The method according to claim 1, wherein the ionic compound comprises one or more cations selected from: 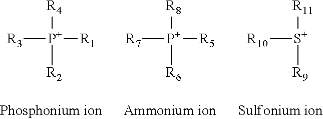
wherein
R1, R2, R3, R4, R5, R6, R7, R8, R9, R10 and R11 can be, independently, hydrogen, alkyl, halogenated alkyl, aminoalkyl, alkenyl, alkynyl, aryl, heteroaryl, cycloalkyl, cycloalkenyl, heterocycloalkyl, or heterocycloalkenyl;
the positively charged P, N and S atoms may individually be part of heterocyclic or heteroaromatic structures by letting:
a two of R1, R2, R3, R4, be fused, or
by letting two of R5, R6, R7, R8 be fused, such that a cyclic ammonium on is formed, such as a pyridinium or imidazolium ion, or,
by letting two of R9, R10 and R11 be fused, such that a cyclic sulfonium on is formed.

4. The method according to claim 1, wherein the thermal degradation temperature of the absorbed ionic compound is higher by Δ_{T} relative to the unsupported, neat ionic compound, and wherein Δ_{T} is at least 5% of the thermal degradation temperature of the unsupported ionic compound.

5. The method according to claim 1, wherein the ionic compound is selected from ammonium carboxylates, imidazolium carboxylates, pyridinium carboxylates, phosphonium carboxylates, ammonium phosphonates, imidazolium phosphonates, pyridinium phosphonates, phosphonium phosphonates, ammonium sulphonates, imidazolium sulphonates, pyridinium sulphonates, phosphonium sulphonates, ammonium acyclovirates, imidazolium acyclovirates and pyridinium acyclovirates.

6. The method according to claim 1, wherein the ionic compound is an ionisable compound selected from carboxylic adds, phosphoric adds and sulphonic adds.

7. The method according to claim 1, wherein the ionic compound is an ionic liquid.

8. The method according to claim 1, wherein the porous solid support is selected from inorganic carrier materials such as silica and mesoporous oxides of niobium, tantalum, titanium, zirconium, cerium and tin, or from carbonaceous or polymeric solids, or from combinations and mixtures thereof.

9. The method according to claim 1, wherein the porous solid support material is silica.

10. The method according to claim 9, wherein the porous solid support material is mesoporous silica having a pore size of between 20-200 Å, provided the solid support does not have a pore size of 90 Å (SiO₂-90).

11. A pharmaceutical, herbicidal or biocidal composition comprising a solid support ionic compound, the solid support ionic compound manufactured by absorbing said ionic compound on a porous solid support material having a pore size of about 20-200 Å, provided the solid support does not have a pore size of 90 Å, said ionic compound comprising an anion that act as a hydrogen ion acceptor.

12. The method according to claim 3, wherein R1 and R2 are fused such that a cyclic phosphonium on is formed or R5 and R6 are fused such that a cyclic ammonium on is formed or R9 and R10 are fused such that a cyclic sulfonium on is formed.

13. The method according to claim 12, wherein R5 and R6 are fused such that a cyclic ammonium on is formed, wherein the cyclic ammonium ion comprises a pyridinium or imidazolium ion.

14. The method according to claim 4, wherein Δ_{T} is at least 10%.

15. The method according to claim 4, wherein Δ_{T} is at least 15%.

16. The method according to claim 4, wherein Δ_{T} is at least 20%.

17. The method according to claim 4, wherein Δ_{T} is at least 25%.

18. The method according to claim 4, wherein Δ_{T} is at least 30%.
